(19) United States
(12) Patent Application Publication
(10) Pub. No.: US 2009/0042205 A1
(43) Pub. Date: Feb. 12, 2009

(54) FLUORESCENCE DETECTION OF DNA BREAKS USING MOLECULAR OSCILLATORS

(75) Inventor: Vladimir Didenko, Houston, TX (US)

Correspondence Address: WINSTEAD PC, P.O. BOX 50784, DALLAS, TX, 75201, US

(73) Assignee: Baylor College of Medicine, Houston, TX (US)

(21) Appl. No.: 12/170,372

(22) Filed: Jul. 9, 2008

Related U.S. Application Data
(60) Provisional application No. 60/948,617, filed on Jul. 9, 2007.

(51) Int. Cl. C12Q 1/68; C12M 1/00; C07H 21/04; G01N 33/00

(52) U.S. Cl. 435/6; 435/287.2; 536/24.3; 436/94

(57) ABSTRACT

A method to detect DNA breaks includes providing a mixture of fluorescence energy transfer molecular oscillators and a DNA sample. The FET oscillator is a synthetic oligonucleotide that has a topoisomerase recognition sequence, a fluorescence donor and a fluorescence acceptor. The synthetic oligonucleotide is bound to a type I topoisomerase capable of binding to the topoisomerase recognition sequence. The mixture is irradiated at a wavelength of the fluorescence donor, and the emission is measured. Another variant of the disclosure is a probe for detecting DNA breaks utilizing a synthetic oligonucleotide comprising a topoisomerase recognition sequence, a fluorescence donor, and a nonradiative fluorescence quencher. Yet another variant of the disclosure is a probe for detecting DNA breaks utilizing a synthetic oligonucleotide comprising a topoisomerase recognition sequence, a fluorescence donor, and a fluorescence acceptor. The mixture is irradiated at a wavelength of the fluorescence donor; and the emission is measured. A method to detect DNA breaks may use these probes in a manner similar to that of the FET oscillator. The FET oscillators and probes are capable of being prepared in a kit formulation.

This application claims priority to U.S. provisional patent application 60/948,617, filed Jul. 9, 2007, which is incorporated by reference as if written herein in its entirety.

### STATEMENT OF FEDERALLY FUNDED RESEARCH

This work was federally sponsored by the National Institute of Health grants NBIB (EB006301) and NINDS (NS054855).

### REFERENCE TO SEQUENCE LISTING

Included with the present specification is a Sequence Listing submitted electronically as a .txt file of 5.92 KB for all oligonucleotide sequences (SEQ ID NOS. 1, and 3-6) and vaccinia virus topoisomerase IB protein (SEQ ID NO. 2). The sequence listing was generated using PatentIn version 3.5 and is incorporated by reference herein in its entirety.

### BACKGROUND

Fluorescence energy transfer (FET) comprises Förster resonance energy transfer (FRET) and non-Förster resonance energy transfer mechanisms between two chromophores. In a typical fluorescence energy transfer experiment, a fluorescent donor is taken to an excited state at a specific fluorescence excitation wavelength. The excited state energy is then nonradiatively transferred to a second molecule, the acceptor, to produce an excited state in the acceptor molecule. Concomitantly, the fluorescence donor returns to the electronic ground state with emission of electromagnetic energy, generally of a longer wavelength than the incident excitation photon. The donor and acceptor entities are capable of being located either in separate molecules or contained as part of the same molecule at two loci.

Fluorescence energy transfer efficiency, between donor and acceptor chromophores in case of nonradiative FET, is generally affected by three parameters: 1) the distance between the donor and the acceptor, the efficiency of nonradiative FET being inversely proportional to the sixth power of the intrachromophore separation, 2) the spectral overlap of the donor emission spectrum and the acceptor absorption spectrum, and 3) the relative orientation of the donor emission dipole moment and the acceptor absorption dipole moment. These three design principles make FET a potentially valuable tool to observe and quantify molecular dynamics in protein cleavage, DNA cleavage, protein-protein interactions, protein-DNA interactions, DNA-DNA interactions, and protein conformational changes. In non-FET based quenching, the spectral overlap of the donor emission spectrum and the acceptor absorption spectrum, and the relative orientation of the donor emission dipole moment and the acceptor absorption dipole moment are of insignificant consequence.

In light of the foregoing, it would be beneficial to develop new systems and methods that use FET and non-FET based quenching in the study of biomolecules.

### SUMMARY

In the most general sense, the present disclosure describes a semi-artificial nanomachine comprised of a first biological molecule and a second biological molecule, wherein the second biological molecule has a complementary sequence for binding of the first biological molecule. Binding of the first biological molecule to the second biological molecule results in dissociation of the nanomachine into smaller working units, which perform an observable function.

In one aspect, the present disclosure provides a method to detect DNA breaks that includes mixing a fluorescence energy transfer (FET) oscillator and a DNA sample. The FET oscillator is comprised of a synthetic oligonucleotide and a type I topoisomerase. The synthetic oligonucleotide is comprised of a topoisomerase recognition sequence, a fluorescence donor, and a fluorescence acceptor. The type I topoisomerase is capable of binding to the topoisomerase recognition sequence in the synthetic oligonucleotide. Upon mixing of the synthetic oligonucleotide and type I topoisomerase, self-assembly to form the FET oscillator occurs. After adding the DNA sample to create a mixture, the mixture is irradiated at an absorption wavelength of the fluorescence donor, and the emission spectrum of the irradiated mixture is measured.

The present disclosure also provides a probe for detecting sequence DNA breaks comprising a synthetic oligonucleotide. The synthetic oligonucleotide is comprised of a topoisomerase recognition sequence, a fluorescence donor, and a nonradiative fluorescence quencher.

The present disclosure also provides another probe for detecting sequence DNA breaks comprising a synthetic oligonucleotide. The synthetic oligonucleotide is comprised of a topoisomerase recognition sequence, a fluorescence donor, and a fluorescence acceptor.

In another aspect of the present disclosure, a method to detect DNA breaks is provided, wherein the DNA breaks are detected utilizing the probe comprised of an oligonucleotide sequence having either a nonradiative fluorescence quencher or a fluorescence acceptor.

In a further aspect, the FET oscillator is capable of being formulated as a kit. The probes comprised of an oligonucleotide having either a nonradiative fluorescence quencher or a fluorescence acceptor may also be formulated as a kit.

The foregoing has outlined rather broadly the features of the present disclosure in order that the detailed description that follows may be better understood. Additional features and advantages of the disclosure will be described hereinafter, which form the subject of the claims.

### BRIEF DESCRIPTION OF THE DRAWINGS

For a more complete understanding of the present disclosure, and the advantages thereof, reference is now made to the following descriptions to be taken in conjunction with the accompanying drawings describing a specific embodiment of the disclosure, wherein:

FIG. 1 shows that bound Vaccinia topoisomerase I of SEQ ID NO. 2 activates the oligonucleotide of SEQ ID NO. 1 toward fluorescence energy transfer by bending the oligonucleotide of SEQ ID NO. 1.

FIG. 2 shows the FET oscillator self-assembles from the oligonucleotide of SEQ ID NO. 1 and Vaccinia topoisomerase I of SEQ ID NO. 2 and oscillates between ligated and cut phases.

FIG. 3 shows the FET-based color shift after detection of 5′-OH blunt-ended DNA by FET oscillators.

FIG. 4 shows FET-based detection of 5′-OH DNA breaks in cell suspension using FET oscillators.

### DETAILED DESCRIPTION

In the following description, certain details are set forth such as specific quantities, sizes, etc. so as to provide a thorough understanding of the present embodiments disclosed herein. However, it will be obvious to those skilled in the art that the present disclosure may be practiced without such specific details. In many cases, details concerning such considerations and the like have been omitted inasmuch as such details are not necessary to obtain a complete understanding of the present disclosure and are within the skills of persons of ordinary skill in the relevant art.

Referring to the drawings in general, it will be understood that the illustrations are for the purpose of describing a particular embodiment of the disclosure and are not intended to be limiting thereto.

While most of the terms used herein will be recognizable to those of skill in the art, the following definitions are nevertheless put forth to aid in the understanding of the present disclosure. It should be understood, however, that when not explicitly defined, terms should be interpreted as adopting a meaning presently accepted by those of skill in the art.

“Biological molecule,” as defined herein, refers to a molecule found in an organism. Examples of biological molecules may include, but are not limited to nucleic acids, DNA, RNA, oligonucleotides, polynucleotides, nucleosides, nucleotides, amino acids, peptides, oligopeptides, polypeptides, proteins, glycoproteins, enzymes, lipids, phospholipids, glycolipids, hormones, peptide hormones, neurotransmitters, carbohydrates, sugars, disaccharides, trisaccharides, oligosaccharides, polysaccharides, antibodies, and antibody fragments. Synthetic derivatives and analogs of biological molecules, including modifications to existing biological molecules or to biological molecules synthesized de novo are encompassed within the term. Synthetic derivatives and analogs of biological molecules either may or may not occur naturally within an organism.

“Chromophore,” as defined herein, refers to a molecule comprising a chemical group that absorbs light at a specific frequency and so imparts color to a molecule.

“Complementary,” as defined herein, refers to a nucleic acid that it is capable of base-pairing with another nucleic acid according to the standard Watson-Crick, Hoogsteen or reverse Hoogsteen binding complementarity rules. It also may refer to a nucleic acid comprising a sequence of consecutive nucleobases or semi-consecutive nucleobases (e.g., one or more nucleobase labels are not present in the molecule) capable of hybridizing to another nucleic acid strand or duplex, even if less than all the nucleobases base pair with a counterpart nucleobase.

“DNA having an end characteristic of apoptosis,” as defined herein, refers to DNA having a ligatable end. Included are ligatable 3′ overhangs, ligatable 5′ overhangs, ligatable 5′ recessed ends, and ligatable blunt ends.

“Fluorophore,” as defined herein, refers to a molecule comprising a chemical group having luminescence resulting from absorption of incident radiation at one wavelength followed by nearly immediate emission of radiation, usually at a different wavelength, that ceases almost at once when the incident radiation stops.

“Ligation,” as defined herein, refers to the process of forming phosphodiester bonds between two nucleic acid or oligonucleotide fragments. To ligate the DNA fragments together, the ends of the DNA fragments must be compatible with each other.

“Nucleic acid,” as defined herein, refers to a molecule comprised of a nucleobase. The term nucleic acid encompasses strands of DNA, RNA, and derivatives or analogs thereof, which comprise a nucleobase. The term nucleic acid also encompasses the terms oligonucleotide and polynucleotide, each as a subgenus of the term nucleic acid. These definitions generally refer to a single-stranded molecule, but in specific embodiments will also encompass an additional strand that is partially, substantially or fully complementary to the single-stranded molecule. Thus, a nucleic acid may encompass a double-stranded molecule or a triple-stranded molecule that comprises one or more complementary strand(s) or complement(s) of a particular sequence comprising a molecule. As used herein, a single stranded nucleic acid may be denoted by the prefix “ss,” a double stranded nucleic acid by the prefix “ds,” and a triple stranded nucleic acid by the prefix “ts.”

“Nucleobase,” as defined herein, refers to a heterocyclic base, such as, for example, a naturally occurring nucleobase (i.e., an A, T, G, C or U) found in at least one naturally occurring nucleic acid (i.e., DNA and RNA), and naturally or non-naturally occurring derivative(s) and analogs of such a nucleobase. A nucleobase generally can form one or more hydrogen bonds (“anneal” or “hybridize”) with at least one naturally occurring nucleobase in a manner that may substitute for naturally occurring nucleobase pairing (e.g., the hydrogen bonding between A and T, G and C, and A and U). One skilled in the art will realize that a nucleobase may be comprised in a nucleoside or nucleotide, using any chemical or natural synthesis method described herein or known to one of ordinary skill in the art.

“Nucleoside,” as defined herein, refers to an individual chemical unit comprising a nucleobase covalently attached to a nucleobase linker moiety. A non-limiting example of a “nucleobase linker moiety” is a sugar comprising 5-carbon atoms (i.e., a “5-carbon sugar”), including but not limited to a deoxyribose, a ribose, an arabinose, or a derivative or an analog of a 5-carbon sugar. Non-limiting examples of a derivative or an analog of a 5-carbon sugar include a 2′-fluoro-2′-deoxyribose or a carbocyclic sugar wherein a carbon atom is substituted for an oxygen atom in the sugar ring. Different types of covalent attachment(s) of a nucleobase to a nucleobase linker moiety are known in the art. By way of non-limiting example, a nucleoside comprising a purine (i.e., A or G) or a 7-deazapurine nucleobase typically covalently attaches the 9-position of a purine or a 7-deazapurine to the 1′-position of a 5-carbon sugar. In another non-limiting example, a nucleoside comprising a pyrimidine nucleobase (i.e., C, T or U) typically covalently attaches the 1-position of a pyrimidine to a 1′-position of a 5-carbon sugar.

“Nucleotide,” as defined herein, refers to a nucleoside further comprising a “backbone moiety.” A backbone moiety generally covalently attaches a nucleotide to another molecule comprising a nucleotide or to another nucleotide to form a nucleic acid. The “backbone moiety” in naturally occurring nucleotides typically comprises a phosphorus moiety, which is covalently attached to a 5-carbon sugar. The attachment of the backbone moiety typically occurs at either the 3′- or 5′-position of the 5-carbon sugar. Other types of attachments are known in the art, particularly when a nucleotide comprises derivatives or analogs of a naturally occurring 5-carbon sugar or phosphorus moiety.

“Nucleic acid analogs,” as defined herein, refers to a derivative or analog of a nucleobase, a nucleobase linker moiety, and/or backbone moiety that may be present in a naturally-occurring nucleic acid. Derivative refers to a chemically modified or altered form of a naturally occurring molecule, while the terms mimic or analog refer to a molecule that may or may not structurally resemble a naturally occurring molecule or moiety, but possesses similar functions. Nucleobase, nucleoside and nucleotide analogs or derivatives are well known in the art, and have been described (see for example, Scheit, 1980, incorporated herein by reference).

“Oligonucleotide,” as defined herein, refers to a molecule of greater than about 3 nucleobases in length.

“Oligonucleotide duplex,” as defined herein, refers to a length of nucleotides wherein each nucleotide is bound to a nucleotide on the opposite strand of DNA.

“Opposite strand of DNA,” as defined herein, refers to DNA that is at least partially complementary to a given length of DNA and may or may not be base paired at each nucleotide or contiguously linked to the given length of DNA by phosphodiester bonds.

“Purine” and/or “pyrimidine,” as defined herein, encompass naturally occurring purine and/or pyrimidine nucleobases and derivative(s) and analog(s) thereof, including but not limited to, purine and/or pyrimidine nucleobase(s) substituted at any chemically compatible point by one or more of an alkyl, carboxyalkyl, aryl, carboxyaryl, amino, alkylamino, dialkylamino, arylamino, diarylamino, arylalkylamino, carboxy, hydroxy, alkoxy, halogen (i.e., fluoro, chloro, bromo, or iodo), thiol, alkylthiol, or arylthiol moiety. The positions at which chemical substitution is feasible with a particular functional group will be evident to those of skill in the art.

“Oscillator,” as generally defined herein, is a system with cyclic variations in output. In one embodiment described herein, the oscillator periodically changes fluorescence output of donor and acceptor chromophores.

Molecular machines and devices assembled from artificial and biological materials belong to the class of semi-artificial nanomachines (Didenko V. V., Minchew C. L., Shuman S., Baskin D. S. Semi-artificial fluorescent molecular machine for DNA damage detection. Nano Letters, 4, 2461-2466; 2004.). Semi-artificial nanomachines and devices harness properties and activity of biological molecules to carry out their function. These nanomachines do not emulate mechanical macromachines or the “scaled-down” approach of mechanical macromachines, which straightforwardly extends the macroscopic concepts of machine motors and parts such as wheels, axles, belts, etc. to the nanoscale constructs (Fabbrizzi, L.; Foti, F.; Licchelli, M.; Maccarini, P. M.; Sacchi, D.; Zema, M. Chemistry 2002, 8, 4965-4972. Drexler, K. E. Nanosystems: Molecular Machinery, Manufacturing and Computation. Wiley: New York, 1992. Freitas, R. A. Nanomedicine: basic capabilities. Landes Bioscience: Austin, 1999.). This concept is one feature that distinguishes semi-artificial nanomachines and devices from related nanoscale machines and devices. Semi-artificial nanomachines and devices may display immediate utility, as they capitalize on the workable mechanisms validated in the evolution of biological molecules.

In the most general aspects, the present disclosure describes a semi-artificial nanomachine comprised of a first biological molecule and a second biological molecule, wherein a complementary sequence is located in one biological molecule for binding of the other biological molecule to it. Binding of the two biological molecules together results in dissociation of the nanomachine into smaller working units which perform an observable function. In other words, Applicant's semi-artificial nanomachine advantageously does not maintain a permanent molecular structure and forms smaller working units after binding of the first biological molecule to the second biological molecule. The first biological molecule and the second biological molecule may be independently chosen to be a naturally occurring biological molecule and/or a synthetic derivative or analog of a naturally occurring biological molecule as provided in the definitions of terms. The binding process can be viewed as providing power to run the semi-artificial nanomachine, in analogy to a motor. In some embodiments, the two biological molecules bind together through a self-assembly process. In a non-limiting example of the semi-artificial nanomachine, the two biological molecules are an oligonucleotide and an enzyme.

The present disclosure provides a topoisomerase-driven fluorescent energy transfer oscillator (hereinafter “FET oscillator”) as one example of a semi-artificial nanomachine. The FET oscillator self-assembles from two molecular components: an oligonucleotide having at least one fluorescent tag and a motor protein. As disclosed herein, a FET oscillator is capable of being used in a method to detect DNA breaks. Such a method begins with providing a mixture to form a FET oscillator comprised of a synthetic oligonucleotide and a type I topoisomerase. The FET oscillator is capable of being comprised of any synthetic oligonucleotide that includes a topoisomerase recognition sequence, a fluorescence donor, and a fluorescence acceptor. To form the FET oscillator, the type I topoisomerase is capable of binding to the topoisomerase recognition sequence. After mixing of the synthetic oligonucleotide and type I topoisomerase, self-assembly of the FET oscillator is allowed to occur. A DNA sample is added to the FET oscillator to create a mixture. The mixture is irradiated at an absorption wavelength of the fluorescence donor, and the emission spectrum is measured. The FET mechanism transfers energy to the fluorescence acceptor, and energy is released as the donor relaxes to ground state. The energy emission spectrum can be observed by routine fluorescence detection, in a non-limiting example.

The FET oscillators of the disclosure rely on mechanisms unique for the nanoenvironment; therefore, unlike “scaled-down” machines, they generally cannot be “scaled-up” to the macroenvironment. Several features keep the design of the semi-artificial nanomachine simple yet functional in the nanoscale environment. One construct is that the FET oscillators are non-motor driven molecular devices, although they use the same force of Brownian motion, which drives directional ratchet mechanisms of molecular motors and pumps. In the traditional sense of molecular devices, the type I topoisomerase of the FET oscillators is analogous the motor element of other molecular devices, even though the method whereby the type I topoisomerase drives the FET oscillators is different. Guided directional motion is not essential for FET oscillators to perform their work, because random movement, including but not limited to Brownian motion, is an efficient mechanism for providing 1) an oscillator restoring force and 2) a surveying means for various molecular targets in solution. In addition the FET oscillator uses both “bottom-up” and “top-down” approaches as it first self-assembles from individual building blocks and then cleaves itself into smaller working units. This “bottom-up-top-down” strategy mimics pathways used by enzymatic systems in vivo and provides simple design and durable performance. No external building blocks or guided assembly are needed for the fabrication of the FET oscillators, and conversion to the working unit is accomplished by a simple enzymatic cleavage. Unlike previously developed constructs of molecular machines, Applicant's FET oscillator advantageously does not maintain a permanent molecular structure as its parts may continuously dissociate and religate. As such, Applicant's semi-artificial nanomachine does not require a “stiff” or rigid frame to fabricate the working units and to withstand persistent molecular collisions.

In one embodiment of the FET oscillator disclosed herein, the synthetic fluorescent oligonucleotide is comprised by a self-complimentary 38-mer core oligonucleotide (SEQ ID NO. 1), which spontaneously folds into a duplex, interrupted by a centrally located nick. In other embodiments, the FET oscillator is comprised by a similar self-complimentary oligonucleotide having SEQ ID NO. 6. One apex of this barbell-shaped structure is labeled with a fluorescein, and the other apex is labeled with a rhodamine. The fluorescein and rhodamine form a donor-acceptor pair which can participate in FET energy transfer. A CCCTT3′ motif, which is a recognition sequence for vaccinia topoisomerase I (SEQ ID NO. 2), is located adjacent to the central nick.

In some embodiments, the oligonucleotide comprising the FET oscillator is capable of being labeled wherein the fluorescence donor is a fluorescein derivative. In further embodiments, the oligonucleotide comprising the FET oscillator is capable of being labeled wherein the fluorescence acceptor is a fluorescein derivative. In another embodiment, the oligonucleotide comprising the FET oscillator is capable of being labeled wherein the fluorescence donor is a rhodamine derivative. In still another embodiment, the oligonucleotide comprising the FET oscillator is capable of being labeled wherein the fluorescence acceptor is a rhodamine derivative. In certain embodiments, the fluorescence acceptor is capable of being a fluorescein derivative and the fluorescence acceptor is capable of being a fluorescein derivative. In other embodiments, the fluorescence donor is capable of being a rhodamine derivative and the fluorescence acceptor is capable of being a rhodamine derivative. In still other embodiments, the fluorescence donor is capable of being a fluorescein derivative and the fluorescence acceptor is capable of being a rhodamine derivative. In still other embodiments, the fluorescence donor is capable of being a rhodamine derivative and the fluorescence acceptor is capable of being a fluorescein derivative. The fluorescence donor or the fluorescence acceptor may alternately be comprised by molecules which are neither fluorescein derivatives nor rhodamine derivatives (non-fluorescein/rhodamine molecules). In some embodiments, the fluorescence donor is capable of being a non-fluorescein/rhodamine molecule, and the fluorescence acceptor is capable of being either a fluorescein derivative or a rhodamine derivative. In some embodiments, the fluorescence donor is capable of being a fluorescein derivative or rhodamine derivative, and the fluorescence acceptor is capable of being a non-fluorescein/rhodamine molecule. In certain aspects of the disclosure, both the fluorescence donor and fluorescence acceptor is capable of being comprised by non-fluorescein/rhodamine molecules. The applicability of a given fluorescent label will vary for a given oligonucleotide and intended application. Choice of a particular fluorescence donor/fluorescence acceptor pair for a given application will be obvious to one skilled in the art. Choice of a given fluorescence donor/fluorescence acceptor pair is not intended to be limiting in the disclosure, and one skilled in the art will recognize that many such fluorescence donor/fluorescence acceptor pairs are capable of being used to operate equivalently within the spirit and scope of the disclosure. In certain embodiments of the disclosure, the fluorescence acceptor is replaced by a molecule that nonradiatively quenches donor fluorescence. Labeling of individual nucleotides comprising the FET oscillator with a fluorescence donor, a fluorescence acceptor, or a fluorescence quencher may occur at the 3′ or 5′ end of the nucleotide or at an internal position of the nucleotide molecule. In certain instances, the fluorescent tag may take the place of an individual nucleotide in the oligonucleotide sequence.

A fluorescein or fluorescein derivative may comprise the fluorescence donor or fluorescence acceptor in any of the embodiments described herein. Non-limiting examples of fluorescein derivatives commonly used in the art may include, but are not limited to, fluorescein, FAM, FITC, TET, DTAF, HEX, JOE, VIC, NED, SNAFL, Alexa dyes, CAL Fluor dyes, DyLight dyes, Oregon Green dyes, Tokyo Green, Yakima Yellow, naphthofluorescein, carboxynaphthofluorescein, and derivatives thereof.

A rhodamine or rhodamine derivative is capabole of comprising the fluorescence donor or fluorescence acceptor in any of the embodiments described herein. Non-limiting examples of rhodamine derivatives commonly used in the art include, but are not limited to, TRITC, TAMRA, TMR, ROX, rhodamine B, rhodamine 6G, carboxyrhodamine 6G, Lissamine Rhodamine B, Rhodamine Red-X, Texas Red, Texas Red-X, QSY dyes, Rhodamine Green, Rhodamin Red, Alexa dyes, CAL Fluor dyes, and derivatives thereof.

Certain other molecules which are neither fluorescein derivatives nor rhodamine derivatives comprise the fluorescence donor or fluorescence acceptor in any of the embodiments described herein. Non-limiting examples of such molecules commonly used in the art may include, but are not limited to, BODIPY, Alexa dyes, CAL Fluor dyes, IRD dyes, Cy dyes, Marina Blue, Pacific Blue, Pacific Orange, Cascade Blue, PyMPO, Cascade Yellow, Dapoxyl, Quasar dyes, Oyster dyes, LC dyes, and derivatives thereof.

Nonradiative fluorescence quenchers may optionally replace the fluorescence acceptor in some embodiments of the disclosure. Non-limiting examples of such molecules commonly used in the art may include, but are not limited to DDQ-I, DDQ-II, Dabcyl, Dansyl, Eclipse, Iowa Black FQ, Iowa Black RQ, BHQ-0, BHQ-1, BHQ-2, BHQ-3, QSY-7, QSY-9, QSY-21, QSY-35, and derivatives thereof.

The motor-protein part of the FET oscillator is comprised by vaccinia DNA topoisomerase I (SEQ ID NO. 2, hereinafter VACC TOPO), a virus-encoded eukaryotic type IB topoisomerase, in an embodiment (Shuman, S. Biochim. Biophys. Acta 1998, 1400, 321-337.). Type IB topoisomerases fall into two highly related but separate structural categories. VACC TOPO (SEQ ID NO. 2) is a 314-amino-acid enzyme containing a small N-terminal domain that promotes DNA binding and increases enzyme processivity. During turnover, VACC TOPO is thought to engage DNA by first wrapping both domains around the DNA duplex like a pair of jaws. The enzyme may then incise one strand and covalently attach to the 3′ side of the break through its catalytic tyrosine residue. Following cleavage, DNA 5′ to the break is allowed to rotate about the intact phosphodiester bond on the uncleaved strand to relax either positively or negatively supercoiled DNA. The DNA is religated and released by the enzyme after one or several DNA turns to complete the reaction. The oscillator design advantageously exploits the ability of VACC TOPO to catalyze rapid and reversible scission and re-joining of DNA stands. Mechanistic details of the role VACC TOPO plays in cleaving DNA should not be considered limiting to the disclosure, and one skilled in the art will recognize that alternative mechanistic explanations may be proposed to describe the function of the enzyme.

The FET molecular oscillator goes through the following cycle of transitions:

### 1. Self-assembly:

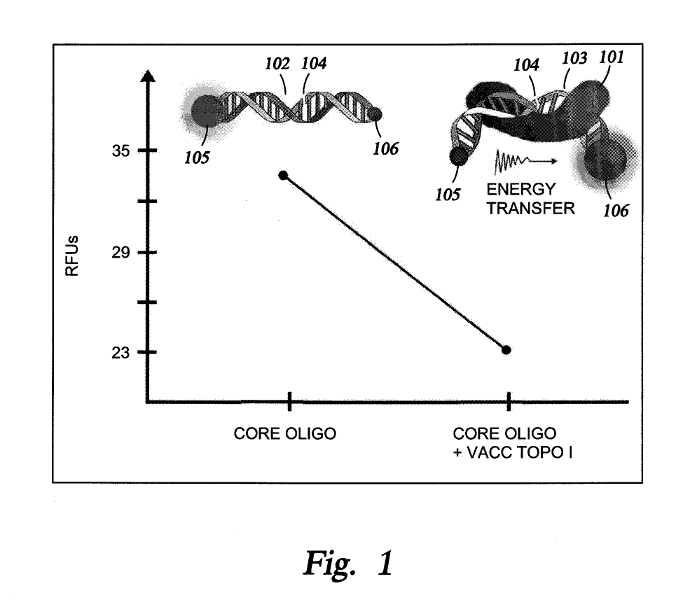

(a) The topoisomerase molecule non-covalently binds to the CCCTT3′ recognition sequence located in the central part of the oligonucleotide occupying nucleotides from −9 to +13 (in relation to the nick position +1) and 13 nucleotides on each side of the nick on the opposite strand (Shuman, S. J. Biol. Chem. 1991, 266, 11372-11379.). The specific binding of the topoisomerase to the CCCTT3′ motif places the enzyme into a lower energy state, keeping it in place until after it has cut the oligonucleotide.
(b) The estimated length of the core oligonucleotide (SEQ ID NO. 1)18 base pair-long stem is about 64.6 Å, which is above the Förster radius (R₀) for the fluorescein-rhodamine pair. The R₀ limit below which FET occurs for this fluorescence-donor pair is about 55 Å (Haugland R. P. Handbook of Fluorescent Probes and Research Products, Ninth Edition, Molecular Probes, Inc. 2002, 964 p.). As shown in FIG. 1, when VACC TOPO 101 binds to the probe duplex 102, it circumferentially encircles it (Sekiguchi, J., Shuman, S. J. Biol. Chem. 1994, 269, 31731-31734.) as a C-shaped protein clamp. Applicant has determined that this bends the core oligonucleotide 103 and brings donor fluorophore 105 and acceptor fluorophore 106 within the Förster radius range, activating FET. The estimated distance between the donor fluorophore and the acceptor fluorophore in the bent core oligonucleotide is about 23.8 Å. The central nick 104, which interrupts the stem in the core oligonucleotide, relaxes the core oligonucleotide structure and makes such bending easier. Analogous DNA bending has been demonstrated for tyrosine recombinases, which are closely related to VACC TOPO (Subramanya, H. S., Arciszewska, L. K., Baker, R. A., Bird, L. E., Sherratt, D. J., Wigley, D. B. (1997) Crystal structure of the site-specific recombinase, XerD. EMBO J. 16, 5178-87.). The enzymes in both cases have very similar catalytic domains and bind to DNA duplexes circumferentially as C-shaped protein clamps (Sekiguchi, J., Shuman, S. (1994) Vaccinia topoisomerase binds circumferentially to DNA. J. Biol. Chem. 269, 31731-31734. Krogh, B. O., and Shuman, S. (2001) Vaccinia Topoisomerase Mutants Illuminate Conformational Changes during Closure of the Protein Clamp and Assembly of a Functional Active Site. J. Biol. Chem. 276, 36091-36099.)). The results shown in FIG. 1 were obtained as follows: 20 fmol of the core oligonucleotide and 20 fmol of VACC TOPO were combined in 15 μL of 33 mM Tris-HCl/16 mM MgCl₂, (pH 7.4). Fluorescence of the core oligonucleotide was measured before and after its reaction with VACC TOPO using a Tecan GENios Plus (excitation at 485 nm and emission at 535 nm). The reaction resulted in suppression of the donor fluorescence due to the bending of the oligonucleotide, which placed the donor and acceptor fluorophores within the Förster radius and activated FET. Similar results are obtained with the FET oscillator system based on SEQ ID NO. 6. These two specific embodiments of FET oscillators are meant to be illustrative of the disclosure. Other FET oscillators are capable of being designed and utilized within the spirit and scope of this disclosure by those skilled in the art.

### 2. Fabrication of the Two Fluorescent Parts:

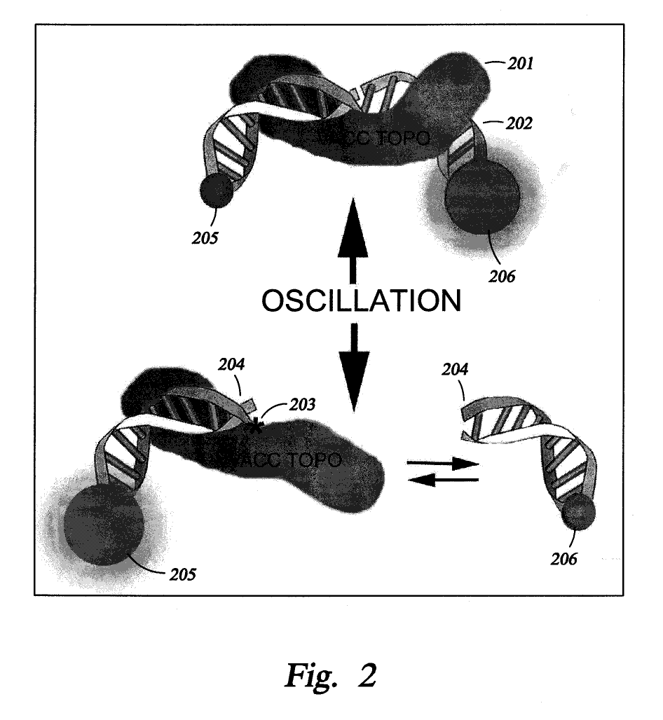

The oscillator operation starts when the topoisomerase molecule 201, bound to the oligonucleotide 202, creates an additional nick 203 at the 3′ end of the CCCTT3′ recognition sequence as shown in FIG. 2 (Shuman, S. S. Site-specific DNA cleavage by vaccinia virus DNA topoisomerase I, J. Biol. Chem. 1991, 266, 1796-1803.). This strand cleavage leads to creation of two separate blunt-ended hairpin DNA fragments 204. The cleaved phosphodiester bond energy is conserved by formation of a covalent link between the 3′ phosphate of the cut DNA strand and a tyrosyl residue of the enzyme (Tyr-274) (Shuman, S.; Kane, E. M.; Morham, S. G. Mapping the active-site tyrosine of vaccinia virus DNA topoisomerase I. Proc. Natl. Acad. Sci. U.S.A. 1989, 86, 9793-9807.). In FIG. 2, the breakage and religation of the core oligonucleotide results in fluorescence of either donor fluorophore 205 (green) or acceptor fluorophore 206 (red). Fluorescent emission of red fluorophore 206 is observed in the religated state, whereas in the cut state fluorescent emission of green fluorophore 205 is observed. The green fluorophore-bearing (205) part, has an activated DNA end (labeled with an asterisk—*) that may react with specific DNA breaks in cells, thus stabilizing green fluorescence.

### 3. Oscillation Between Ligated and Cut States:

(a) The free energy gain for the breakage reaction is small, on the order of about +1 kcal/mole, making the reaction freely reversible (Champoux, J. J. In DNA topology and its biological effects. Cozzarelli, N. R., Wang, J. C. Eds.; Cold Spring Harbor Laboratory Press: Cold Spring Harbor, 1990; pp 217-242.). Therefore, VACC TOPO, which remains bound to the CCCTT motif located on the downstream hairpin, will religate the two hairpins from the cutting process. This leads to cycles of self-attachment-disattachment of the fluorescent hairpins as thermal motion randomly separates and brings together enzyme-bearing and enzyme-free oligonucleotides, which are repeatedly religated and then re-cut by the topoisomerase. This motif possesses features of oscillating systems:
1. Presence of energy that maintains motion needed for restoring force (thermal energy in this case);
2. Exchange between kinetic and potential energy due to the restoring force (i.e., kinetic energy of random motion of separated DNA hairpins and potential energy of their mutual positions); and
3. Existence of equilibrium where forward rate equals the backward rate (the cleavage-religation equilibrium).
(b) The molecular FET oscillator operates after macroscopic equilibrium has been reached, because individual molecules continue to bind and dissociate under the equilibrium conditions. Thus, the operation of a molecular FET oscillator is unlike a macroscopic chemical reaction that “stops” when equilibrium is reached (Schneider, T. D. J. Theor. Biol. 1991, 148, 83-123.). The periodic changes in the mechanical plane manifest in oscillating FET fluorescence outputs from individual donor- and acceptor-bearing hairpins.
(c) In the re-ligated state, the fluorescence of the donor fluorophore is suppressed and fluorescence of the acceptor fluorophore is enhanced due to FET. After cleavage the hairpins separate, thereby ending their energy transfer. This activates the donor fluorescence and quenches the acceptor. As such, in an embodiment, a full cycle for each fluorophore in the FET oscillator comprises two phases: radiative and nonradiative, with donor and acceptor groups fluorescing in counterphase.
(d) The oscillation period indicates the time of an embodiment's complete oscillation cycle and in the FET oscillator is comprised by radiative and nonradiative phases. In the mechanical plane these phases are embodied by the ligated and cleaved states of the core oligonucleotide. Consequently, the length of each phase is an inverse of turnover number (k_{cat}) for either religation or cleavage. Single-turnover and steady-state kinetic measurements and DNA binding studies with DNA duplexes have determined the individual rate constants for strand cleavage mediated by VACC TOPO k_{cl}=0.07 s⁻¹ and religation as VACC TOPO kᵣ=0.66 s⁻¹ (Stivers J. T., Shuman S., Mildvan A. S. Vaccinia DNA topoisomerase I: single-turnover and steady-state kinetic analysis of the DNA strand cleavage and ligation reactions. Biochemistry, 1994; 33:327-39.). The individual rate constants for strand cleavage and religation measurements were performed in these studies using binding of an 8-mer acceptor strand to an oligonucleotide duplex with an 8-base overhang. Although the oligonucleotide is different in the rate measurement system, the ratio k_{cl}/kᵣ≈0.1 refers to the properties of VACC TOPO and not to the oligonucleotide structure. This makes the rate ratio the same as in the Applicant's system.
(e) The ratio of cleavage-religation rate constants at equilibrium favors the uncleaved DNA-enzyme complex by ˜10-fold, as the individual rate constant for DNA religation is an order of magnitude greater than the rate constant for DNA cleavage (Stivers J. T., Shuman S., Mildvan A. S. Vaccinia DNA topoisomerase I: single-turnover and steady-state kinetic analysis of the DNA strand cleavage and ligation reactions. Biochemistry, 1994; 33:327-39.). Consequently, the core oligonucleotide spends most of its time uncleaved, with only brief periods of cleavage before rapid religation back into the uncleaved state with the ratio of fluorescence emission phases φ_{acceptor}/φ_{donors}≈10. Therefore, for most of the time, donor fluorescence is quenched, and the donor becomes radiative only for a brief duration in the cleaved state. This “blinking” system comprises a sensitive detector of DNA breaks.

### 4. Labeling of DNA Breaks:

(a) The cleavage of the dual-labeled oligonucleotide creates two blunt-ended hairpins. In the presence of another DNA acceptor, such as a blunt-ended DNA break with a 5′ OH, the topoisomerase-carrying hairpin will ligate to the DNA acceptor instead of its partner blunt-ended hairpin, fluorescently labeling the DNA acceptor in the process. This interrupts FET energy transfer and stabilizes the donor fluorophore in the radiative phase. The act of DNA break detection stops oscillations and thereby halts energy transfer between donor and acceptor fluorophores. Both spectroscopically active parts of the FET oscillator become essentially permanently separated after one blunt end is bound to a DNA break. This process unmasks the donor fluorescence, quenches the acceptor and provides a signal for detection of the presence of DNA breaks. In other words the donor fluorescence emission spectrum is cloaked unless the oscillator detects its target DNA breaks. This property underlies a utility of the FET oscillator detection of DNA breaks in a homogenous “closed tube” assay format, where no additional interference beyond adding the FET oscillator is needed.
(b) Using the Applicant's FET oscillator system with a confocal microscope setting makes possible detection of DNA breaks on the level of a single break (Byassee T. A.; Chan, W. C.; Nie, S. Anal Chem. 2000, 72, 5606-5611.), because each labeling event places a single fluorophore at the end of a DNA break. The dynamics of DNA break generation can also be observed in such a setting in real time because the fluorescent signal intensity is directly proportional to the number of breaks detected. These properties make the Applicant's system applicable for studying conditions with solitary breaks per genome, such as those induced by low intensity radiation and free radical damage. Such conditions are encountered in space flights or induced by stress or aging, in non-limiting examples. In a broader but still non-limiting application the FET oscillator may be useful in detection of individual cells in early apoptosis as the number of breaks in an apoptotic cell rises from about 50,000 per genome at the initial high molecular weight DNA degradation, to about 3×10⁶ per genome during internucleosomal DNA fragmentation (Walker, P. R.; Leblanc, J.; Carson, C.; Ribecco, M.; and Sikorska, M. Annals Ny Acad. Sci. 1999, 887, 48-59.). The Applicant's FET oscillator is further advantageous in that it exclusively detects double strand blunt-ended DNA breaks with 5′ hydroxyl groups. Although the DNA breaks produced during apoptosis are also double strand and blunt ended, they often carry phosphate groups at the 5′ ends. The 5′ phosphate groups are rapidly cleaved to provide 5′OH groups through the action of cellular kinases and phosphatases, making the DNA breaks detectable by Applicant's FET oscillator.

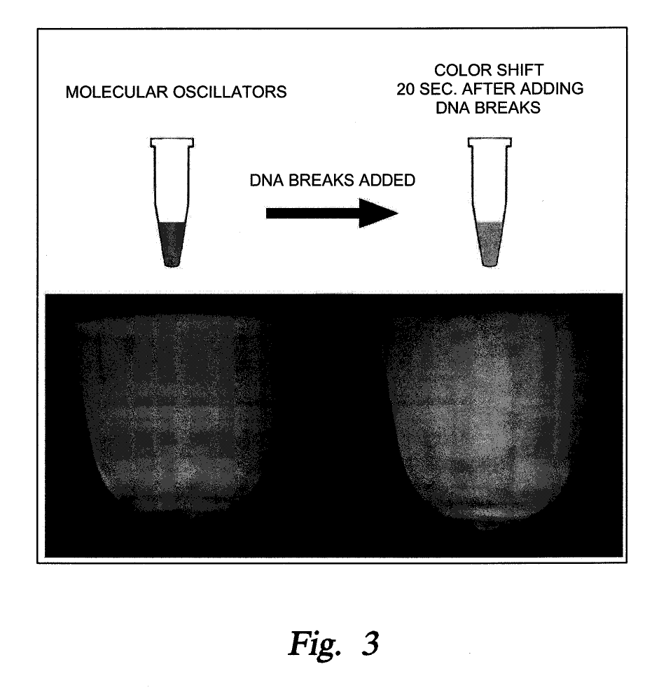

Applicant has performed ultra-fast detection of 5′OH blunt-ended DNA breaks in a model system using various embodiments of a FET molecular oscillator as shown in FIG. 3. Without DNA breaks, the individual oscillators spend most of their time in the religated phase with a nonradiative donor and radiative acceptor (red fluorescence). The oscillators only briefly pass through the cut phase where the donor emits a short burst of green light before reverting to the nonradiative state upon religation. Therefore, the overall fluorescence output is primarily generated by rhodamine acceptor emission (red spectrum) despite illumination at the donor fluorescein excitation wavelength (green spectrum). After DNA breaks (blunt-ended DNA duplexes with a 5′OH) were added, the FET energy transfer between fluorophores stopped or significantly slowed down, resulting in unmasking of the green donor spectrum and suppression of the red acceptor spectrum. This action produced the red to green fluorescence shift as demonstrated in FIG. 3 in a reaction that took 20 seconds at room temperature (23° C.). The images of the tubes containing FET oscillators in FIG. 3 were taken through the objective of a fluorescent microscope under 494 nm (green) excitation light. The left tube (red fluorescence) solution contains a FET oscillator dual labeled with FITC and TAMRA (1 pmol/μL in 50 mM Tris-HCl, pH 7.4) and VACC TOPO (10 pmol/μL). The right tube (green fluorescence) is the same solution 20 seconds after addition of blunt-ended DNA (concentration of DNA ends=10 pmol/μL).

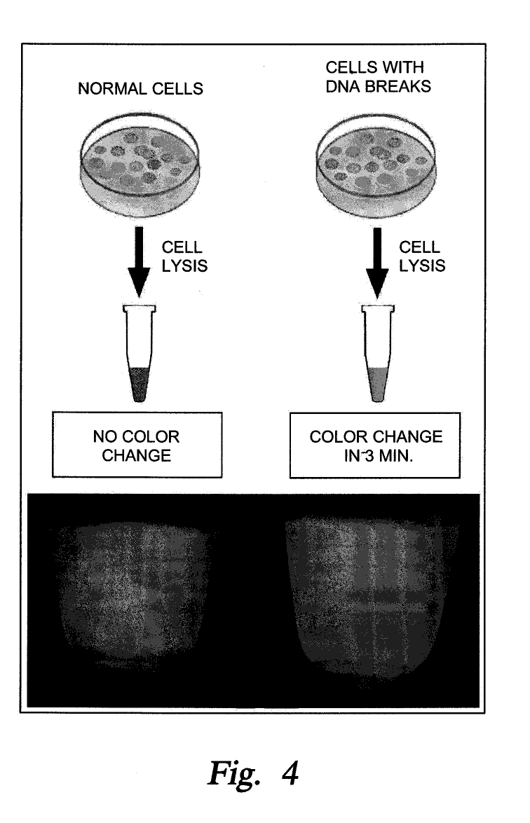

Applicant has used FET oscillators to perform detection of double strand 5′OH DNA breaks produced by treating cultured B-cells with exogenous DNase II. The detection was complete in 3 minutes as signaled by the shift from red to green fluorescence as shown in FIG. 4. The reaction was specific to 5′OH DNA breaks and was unaffected by other types of DNA damage or cellular debris. Although cellular nucleases released during cell lysis could, in theory, contribute to the FET shift via nonspecific cleavage of the FET oscillator, this did not occur as demonstrated by the normal cell control as shown in FIG. 4. The topoisomerase attached to the recognition sequence covers the oscillator oligonucleotide, preventing nuclease damage to the oligonucleotide. In addition, the short hairpin-shaped GC-rich oligonucleotides, which Applicant used, are very poor substrates for cellular nucleases. Such nucleases have a high propensity for longer genomic DNA, especially that rich in AT regions. Furthermore, the probe signal was analyzed after only 3 minutes of incubation, which is long enough for a high speed topoisomerase reaction to occur, but is insufficient for much slower nuclease reactions. Both DNase I and DNase II-type nucleases require a much longer time to produce substantial cleavage, which Applicant has verified (Didenko V. V., Ngo H., Baskin D. S. In situ detection of double-strand DNA breaks with terminal 5′OH groups. In Didenko V. V. (ed) In Situ Detection of DNA Damage: Methods and Protocols, Humana Press, Totowa, N.J., (2002).). In summary, the FET molecular oscillators of this disclosure are not influenced by non-specific DNA damage or cellular components, are not damaged by cellular nucleases, and may rapidly detect low concentrations of cells with DNA breaks. FIG. 4 shows images of tubes containing molecular FET oscillators with cells taken through the objective of a fluorescent microscope under 494 nm (green) excitation light. The left tube (red fluorescence) is a control solution containing FET oscillators dual labeled with FITC and TAMRA (1 pmol/μL in 50 mM Tris-HCl, pH 7.4) and VACC TOPO (10 pmol/μL) along with normal cells lacking 5′OH DNA breaks. The right tube (green fluorescence) contains the same FET oscillators after the addition of cells having 5′OH DNA breaks. The concentration of cells in both tubes is about 5000 cells/μL). The fluorescence color shift in the right tube occurred 3 minutes after addition of the cells having DNA breaks.

Applicant has performed detection of apoptotic cells in a cultured live Jurkat cell suspension. FET oscillators were used in this analysis at 2 fMol/μL in 100 mM Tris-HCl (pH 7.4). Normal cells not having blunt-ended DNA breaks were used as a control. Apoptosis was induced by the agonistic anti-Fas antibody CD95/APO-1 (Biosource; Camarillo, Calif.) and verified morphologically. The apoptotic and control cells were placed in a hypo-osmotic solution and vortexed to rupture cellular membranes. The final concentrations of cells was either 2 or 20 cells/μL (20 and 200 cells per single cell plate well). Following vortexing, the solutions were immediately added to the FET molecular oscillator solution. Green donor fluorescence was observed for the apoptotic cells 3 minutes after addition using a Tecan GENios Plus spectrofluorimeter at 485 nm excitation and 535 nm emission wavelengths. In contrast, red acceptor emission was observed in the control cells. This result indicates the presence of DNA breaks in the apoptotic but not normal cells. The reaction resulted in a 23% increase of 535 nm fluorescence emission in 3 minutes, indicating detection of minimally 2 apoptotic cells/μL (10⁷ breaks/μL). This assay significantly exceeds the speeds of the other currently used approaches for assaying of DNA breaks (see van Dierendonck, J. H. DNA damage detection using DNA Polymerase I or its Klenow fragment: Applicability, Specificity, Limitations. In Didenko V. V. (ed) In Situ Detection of DNA Damage: Methods and Protocols, Humana Press, Totowa, N.J., (2002); Walker P. R., Carson C., Leblanc J., Sikorska M. Labeling DNA damage with Terminal Transferase: Applicability, Specificity, and Limitations. In Didenko V. V. (ed) In Situ Detection of DNA Damage: Methods and Protocols, Humana Press, Totowa, N.J., (2002); Hornsby P. J., Didenko V. V. In situ ligation as a method for labeling apoptotic cells in tissue sections: an overview. In Didenko V. V. (ed) In Situ Detection of DNA Damage: Methods and Protocols, Humana Press, Totowa, N.J., (2002); Didenko V. V., Ngo H., Minchew C., Boudreaux D. J., Widmayer M. A., Baskin D. S. Visualization of irreparable ischemic damage in brain by selective labeling of double strand blunt-ended DNA breaks. Molec Med, 8:818-823 (2002); Venues, I., C. Haanen, H. Steffens-Nakken, and C. Reutelingsperger. 1995. A novel assay for apoptosis. Flow cytometric detection of phosphatidylserine expression on early apoptotic cells using fluorescein labelled Annexin V. J. Immunol. Meth. 184:39-51; Amstad, P. A., G. L. Johnson, B. W. Lee and S. Dhawan. 2000. An in situ marker for the detection of activated caspases. Biotechnology Laboratory 18: 52-56.).

In various alternate embodiments, a FET oscillator assembly is capable of being replaced by a probe for detecting DNA breaks comprised of a fluorescence donor and a nonradiative fluorescence quencher. In various embodiments, the fluorescence quencher takes the place of the fluorescence acceptor in the FET oscillator, but the operational details of the detection system remain essentially the same as in the FET oscillator. In such embodiments, non-Förster based energy transfer takes place. The probe is capable of comprising a synthetic oligonucleotide that is itself comprised by a topoisomerase recognition sequence, a fluorescence donor, and a nonradiative fluorescence quencher. The probe is capable of being further comprised and self-assembled in an analogous manner to that described hereinabove with a type I topoisomerase capable of binding to the topoisomerase recognition sequence. In various embodiments, the type I topoisomerase is a virus-encoded eukaryotic type 1B topoisomerase having SEQ ID NO. 2.

The fluorescence donor in the nonradiative quencher probe is capable of being comprised by any fluorescent tag known to those of skill in the art, including but not limited to fluorescein, FAM, FITC, TET, DTAF, HEX, JOE, VIC, NED, SNAFL, Alexa dyes, CAL Fluor dyes, DyLight dyes, Oregon Green dyes, Tokyo Green, Yakima Yellow, naphthofluorescein, carboxynaphthofluorescein, TRITC, TAMRA, TMR, ROX, rhodamine B, rhodamine-6G, carboxyrhodamine-6G, Lissamine Rhodamine B, Rhodamine Red-X, Texas Red, Texas Red-X, QSY dyes, Rhodamine Green, Rhodamin Red, BODIPY derivatives, IRD dyes, Cy dyes, Marina Blue, Pacific Blue, Pacific Orange, Cascade Blue, PyMPO, Cascade Yellow, Dapoxyl, Quasar dyes, Oyster dyes, LC dyes, and derivatives thereof. The fluorescence quencher in the nonradiative quencher probe is capable of being comprised by any nonradiative quencher known to those of skill in the art, including but not limited to, DDQ-I, DDQ-II, Dabcyl, Dansyl, Eclipse, Iowa Black FQ, Iowa Black RQ, BHQ-0, BHQ-1, BHQ-2, BHQ-3, QSY-7, QSY-9, QSY-21, QSY-35, and derivatives thereof. One skilled in the art will recognize that certain fluorescence donor and quencher pairs are capable of being advantageous for a given application, and that these examples represent non-limiting exemplary suggestions.

In various embodiments, the probe having the fluorescent donor and nonradiative fluorescence quencher has an oligonucleotide component having SEQ ID NO. 4. The probe based on SEQ ID NO. 4 displays photoemission prior to binding the topoisomerase, but no further fluorescence occurs after topoisomerase binding until the probe detects its target. Otherwise, the topoisomerase-bound oligonucleotide SEQ ID NO. 4 is completely nonradiative due to the fluorescence donor and nonradiative quencher residing within the Förster radius. Once the fluorophore-tagged oligonucleotide portion of SEQ ID NO. 4 ligates a DNA break, the fluorescence donor portion of the oligonucleotide is liberated from its nonradiative fluorescence quencher, and photoemission is once again observed.

The probe for detecting DNA breaks may alternately be comprised by a fluorescence acceptor instead of, or in addition to, a nonradiative quencher. The fluorescence acceptor probe is comprised by a synthetic oligonucleotide that is itself comprised by a topoisomerase recognition sequence, a fluorescence donor, and a fluorescence acceptor. This probe may embody many features found in a FET oscillator oligonucleotide, but in certain embodiments the physical location of the fluorescence donor and fluorescence acceptor in the oligonucleotide renders the fluorescence donor nonradiative, even prior to topoisomerase binding. The probe is capable of being further comprised and self-assembled in an analogous manner to that described hereinabove with a type I topoisomerase capable of binding to the topoisomerase recognition sequence. In certain embodiments, the type I topoisomerase is a virus-encoded eukaryotic type 1B topoisomerase having SEQ ID NO. 2.

The fluorescence donor and the fluorescence acceptor in the fluorescence acceptor probe is capable of being comprised by any fluorescent tag known to those of skill in the art, wherein the donor and acceptor entities are independently selected from the group including but not limited to fluorescein, FAM, FITC, TET, DTAF, HEX, JOE, VIC, NED, SNAFL, Alexa dyes, CAL Fluor dyes, DyLight dyes, Oregon Green dyes, Tokyo Green, Yakima Yellow, naphthofluorescein, carboxynaphthofluorescein, TRITC, TAMRA, TMR, ROX, rhodamine B, rhodamine-6G, carboxyrhodamine-6G, Lissamine Rhodamine B, Rhodamine Red-X, Texas Red, Texas Red-X, QSY dyes, Rhodamine Green, Rhodamin Red, BODIPY derivatives, IRD dyes, Cy dyes, Marina Blue, Pacific Blue, Pacific Orange, Cascade Blue, PyMPO, Cascade Yellow, Dapoxyl, Quasar dyes, Oyster dyes, LC dyes, and derivatives thereof.

In an embodiment of the probe having the fluorescence donor and fluorescence acceptor, the oligonucleotide component has SEQ ID NO. 5. The probe based on SEQ ID NO. 5 functions slightly differently than that based on SEQ ID NO. 4. The short probe based on oligonucleotide SEQ ID NO. 5 is nonradiative even prior to binding with the topoisomerase, since its fluorescence donor and fluorescence acceptor are already within the Förster radius. The longer probe having SEQ ID NO. 4 is radiative prior to binding with the topoisomerase, since its fluorescence donor and nonradiative quencher are initially outside the Förster radius. Only after the probe based on SEQ ID NO. 5 dissociates and binds to its target is fluorescence observed, since the fluorescence donor and fluorescence acceptor are separated at that point. The probe based on SEQ ID NO. 5 may be advantageous in applications where a low fluorescence background is beneficial. Embodiments of probes based on SEQ ID NO. 4 and SEQ ID NO. 5 are meant to be illustrative of the disclosure. Other probes utilizing like methodology are capable of being designed and utilized within the spirit and scope of the practice of this disclosure.

A method to detect DNA breaks using either the probe having a fluorescence donor and nonradiative fluorescence quencher or the probe having a fluorescence donor and fluorescence acceptor is provided in the disclosure. The method of detecting DNA breaks is comprised by providing the probe, providing the probe with a DNA sample, irradiating the mixture of the probe and DNA sample at an absorption wavelength of the probe's fluorescence donor, and measuring the emission spectrum of the irradiated mixture. When the disclosure is practiced in this manner, no emission signal from the fluorescence donor is observed when the fluorescence donor oligonucleotide fragment and the fluorescence quencher/fluorescence donor oligonucleotide fragments are ligated to each other. Introduction of DNA breaks allows separation of the donor from the quencher/acceptor and subsequent detection of fluorescence emission of the donor. Observation of fluorescence emission is an observable test for the presence of DNA breaks. One skilled in the art will recognize that the wavelength of fluorescence emission will be determined by the identity of the fluorescence donor entity chosen.

The fluorescence donor or fluorescence acceptor in a method utilizing the probe to detect DNA breaks is capable of being comprised by any fluorescent tag known to those of skill in the art, including but not limited to fluorescein, FAM, FITC, TET, DTAF, HEX, JOE, VIC, NED, SNAFL, Alexa dyes, CAL Fluor dyes, DyLight dyes, Oregon Green dyes, Tokyo Green, Yakima Yellow, naphthofluorescein, carboxynaphthofluorescein, TRITC, TAMRA, TMR, ROX, rhodamine B, rhodamine-6G, carboxyrhodamine-6G, Lissamine Rhodamine B, Rhodamine Red-X, Texas Red, Texas Red-X, QSY dyes, Rhodamine Green, Rhodamin Red, BODIPY derivatives, IRD dyes, Cy dyes, Marina Blue, Pacific Blue, Pacific Orange, Cascade Blue, PyMPO, Cascade Yellow, Dapoxyl, Quasar dyes, Oyster dyes, LC dyes, and derivatives thereof. The fluorescence quencher in the method using the probe to detect DNA breaks is capable of being comprised by any nonradiative quencher known to those of skill in the art, including but not limited to, DDQ-I, DDQ-II, Dabcyl, Dansyl, Eclipse, Iowa Black FQ, Iowa Black RQ, BHQ-0, BHQ-1, BHQ-2, BHQ-3, QSY-7, QSY-9, QSY-21, QSY-35, and derivatives thereof. One skilled in the art will recognize that certain fluorescence donor and quencher/acceptor pairs may be advantageous for a given application, and that these examples represent non-limiting exemplary suggestions.

Finally, it is expected that the FET oscillator and/or the probes described hereinabove are capable of being made available in the form of a kit. In a non-limiting example, the FET oscillator or probes for detecting DNA breaks are formulated either as a solid or solution into a container, which maintains stability of the FET oscillator or probes during storage until the kit is needed for use. Alternately, the oligonucleotide sequence and topoisomerase components are capable of being packaged in separate containers and combined to formulate the kit just prior to use. The kit may then be exposed to a DNA sample and the results of the kit analysis determined using methods described hereinabove. For example, detection is capable of being conducted by spectrophotometric, calorimetric, or conductimetric analyses. Such a kit formulation is capable of being useful for on-site field analysis of DNA.

Accordingly, various embodiments of the present invention disclose semi-artificial nanomachines, the nanomachines comprising a first biological molecule and a second biological molecule, wherein said second biological molecule comprises a complementary sequence for binding of said first biological molecule, wherein binding of said first biological molecule to said second biological molecule results in dissociation of the nanomachine into smaller working units, and wherein said smaller working units perform an observable function. In various embodiments, the first biological molecule and second biological molecule bind together by self-assembly.

Various further embodiments disclose A method to detect deoxyribonucleic acid (DNA) breaks, said method comprising the steps of a) providing a synthetic oligonucleotide comprising a topoisomerase recognition sequence; a fluorescence donor; and, a fluorescence acceptor; b) providing a type I topoisomerase capable of binding said topoisomerase recognition sequence; c) mixing said synthetic oligonucleotide and said topoisomerase and allowing self-assembly of a fluorescence energy transfer (FET) oscillator to occur; d) adding a DNA sample to the FET oscillator to create a mixture; e) irradiating said mixture at an absorption wavelength of said fluorescence donor; and, f) measuring the emission spectrum of said irradiated mixture. In various embodiments, the fluorescence acceptor comprises a nonradiative fluorescence quencher. In various embodiments, the synthetic oligonucleotide comprises a nucleotide sequence corresponding to SEQ ID NO. 1. In various embodiments, the synthetic oligonucleotide comprises a nucleotide sequence corresponding to SEQ ID NO. 6. In various embodiments, the type 1 topoisomerase is a virus-encoded eukaryotic type IB topoisomerase, such as a peptide sequence corresponding to SEQ ID NO. 2.

Various further embodiments disclose probes for detecting deoxyribonucleic acid (DNA) sequence breaks, said probe comprising a synthetic oligonucleotide comprising a topoisomerase recognition sequence; a fluorescence donor; and, a nonradiative fluorescence quencher. In various embodiments, the binding results in self-assembly. In various embodiments, the type I topoisomerase is a virus-encoded eukaryotic type 1B topoisomerase comprising a peptide sequence corresponding to SEQ ID NO. 2. In various embodiments, the synthetic oligonucleotide sequence comprises a nucleotide sequence corresponding to SEQ ID NO. 4.

Various further embodiments disclose a probe for detecting deoxyribonucleic acid (DNA) sequence breaks, said probe comprising a synthetic oligonucleotide comprising a topoisomerase recognition sequence; a fluorescence donor; and, a fluorescence acceptor. In various embodiments, the binding results in self-assembly. In various embodiments, the type 1 topoisomerase is a virus-encoded eukaryotic type 1B topoisomerase comprising a peptide sequence corresponding to SEQ ID NO. 2. In various embodiments, the synthetic oligonucleotide sequence comprises a nucleotide sequence corresponding to SEQ ID NO. 5.

Various further embodiments disclose methods to detect deoxyribonucleic acid (DNA) breaks, said method comprising the steps of providing the probe as herein disclosed; providing a DNA sample; irradiating the mixture of said probe and said DNA sample at an absorption wavelength of said fluorescence donor; and, measuring the emission spectrum of said irradiated mixture.

Various embodiments disclose kits for detecting DNA breaks comprising at least one FET oscillator as disclosed herein. Further, various embodiments disclose a kit for detecting DNA breaks comprising the probe as herein disclosed.

### EXAMPLES

The following examples are included to demonstrate particular embodiments of the present disclosure. It should be appreciated by those of skill in the art that the methods disclosed in the examples that follow merely represent exemplary embodiments of the present disclosure. Those of skill in the art should, in light of the present disclosure, appreciate that many changes can be made in the specific embodiments described and still obtain a like or similar result without departing from the spirit and scope of the present disclosure.

### Oligonucleotides

All oligonucleotides were synthesized by Integrated DNA Technologies, (IDT, Coralville, Iowa). Modified nucleotides are shown in parentheses in the examples below. The modification and the nucleotide being modified are separated by a forward slash (/). For 5′ modifications, the modification precedes the nucleotide, and for 3′ modifications, the modification follows the nucleotide. For internal modifications, the modification precedes the nucleotide, and the modification is denoted with an ‘i’ designation. For example, (FAM/A) is a 3′FAM modified adenosine, and (iFluor/T) is an internally fluorescein labeled thymidine. For modifications where a specific oligonucleotide is implied by the label utilized, the specific nucleotide has been added to the label name for clarity purposes. For example, (i6-TAMN) denotes a TAMRA labeled thymidine. Hereinbelow this labeled nucleotide is denoted (i6-TAMN/T) for clarity; only one thymidine is indicated by this label designation.

(SEQ ID NO. 1)

5′-AAGGGACCTGCFGCAGGTCCCTTAACGCATRATGCGTT-3′

SEQ ID NO. 1 is a double-hairpin vaccinia topoisomerase I cleavable oligonucleotide, dual labeled with fluorescein and rhodamine. F is an internal fluorescein labeled thymidine (fluorescein-dT). R is an internal rhodamine labeled thymidine (rhodamine-dT).

| | |
|---|---|
| 5′-GCGCTAGACCTGGTCTAGCGC-3′ | (SEQ ID NO. 3) |

SEQ ID NO. 3 is a test oligonucleotide with a hairpin as the source of blunt ends for DNA detection tests in solution.

(SEQ ID NO. 4)

5′-(6-FAM/A)AG GGA CCT CTG AGG TCC CTT ACG CAT TAT
GCG (T/BHQ_1)-3′

SEQ ID NO. 4 contains a fluorescence quencher (or dark quencher). The fluorescence quencher (BHQ type 1) is located on the 3′ position of thymidine. The fluorescent FAM tag is a 5′ 6-FAM adenosine. BHQ-1 is a recommended quencher for FAM. SEQ ID NO. 4 displays photoemission prior to binding topoisomerase, but no further fluorescence occurs after binding until the oligonucleotide detects its target. One skilled in the art will recognize that the actual fluorophore pair, or quencher/fluorophore pair, is not essential and many others pairings may participate in this type of interaction.

(SEQ ID NO. 5)

5′-AAG GGA CT(iFluor/T) AGT CCC TTA CGA TT
(i6TAMN/T) ATC GT-3′

SEQ ID NO. 5 is a short probe designated VACC R C Dual Sh. The short probe has a FAM/TAMRA NHS pair. Since the modifications of this probe are internal, a different type of label is used. The iFluor/T notation is used by IDT (Coralville, Iowa) to signify an internal fluorescein labeled dT. The same 6-FAM derivative found in SEQ ID NO. 4 is not available for internal modifications. The internal thymidine TAMRA label is introduced by an NHS ester. An advantage of this short probe is that the donor is nonradiative even prior to reaction with topoisomerase, unlike the longer probe of SEQ ID NO. 4.

(SEQ ID NO. 6)

5′-AAG GG(i6-TAMN/T) CCT GCT GCA GGA CCC TTA ACG
CAT TAT GCG (iFluor/T)T-3′

SEQ ID NO. 6 is a double-hairpin vaccinia topoisomerase I cleavable oligonucleotide, dual labeled with fluorescein and rhodamine. The rhodamine label is an internal TAMRA labeled thymidine introduced via an NHS ester. The fluorescein label is an internal labeled thymidine dT.

### Vaccinia Topoisomerase I

Vaccinia DNA topoisomerase I (SEQ ID NO. 2) was purchased from Chemicon. The oscillating system self-assembles when the Core Oligo and VACC TOPO are combined in the solution of 100 mM Tris-HCl (pH 7.2) at a 1:1 ratio.

### Example 1

### Detection of DNA Breaks in Solution

The FET oscillators were assembled in 50 mM Tris-HCl (pH 7.4) by combining 1 pmol/μL of the oligonucleotide of SEQ ID NO. 1 and 10 pmol/μL vaccinia topoisomerase I (SEQ ID NO. 2). The oligonucleotide of SEQ ID NO. 3 at a concentration of 10 pmol/μL DNA ends in 50 mM Tris-HCl (pH 7.4), was added to the solution and used to emulate blunt-ended DNA breaks. The mixture was illuminated with 494 nm excitation light, and images of the reaction tubes were taken through the objective of a fluorescent microscope. Experimental output from this example is shown in FIG. 3.

### Example 2

### Detection DNase II-Induced DNA Breaks in Suspension of B-Cells

Breaks in these cells were generated by DNase II treatment in hypo-osmotic DNase II buffer for 2 hours, and cell suspensions were subsequently diluted in water to rupture cellular membranes. The DNA breaks generated as such were immediately added to a solution containing FET oscillators formed from the oligonucleotide of SEQ ID NO. 1 and topoisomerase SEQ ID NO. 2 to provide a final concentration of about 5000 cells/μL. Experimental output from this example is shown in FIG. 4. Control cells were either not treated with DNase II and did not have DNA breaks, or were treated with DNase I and did not have specific 5′OH breaks (latter control not shown). A FET-based fluorescence shift in the cells having DNA breaks was detected 3 minutes after the addition of cells to the FET oscillator solution. In contrast, no fluorescence shift was seen in the control solution. Detection was performed using a Tecan GENios Plus spectrofluorimeter at 485 nm excitation and 535 nm emission wavelengths.

### Example 3

### Detection DNA Breaks in Suspension of Apoptotic Jurkat Cells

Apoptosis in cultured Jurkat cells was induced by 5 hours of exposure to the agonistic anti-Fas antibody CD95/APO-1 (Biosource; Camarillo, Calif.) and verified morphologically by DAPI staining. The cells were placed in a hypo-osmotic solution and vortexed to rupture cellular membranes. Two final concentrations of either 2 or 20 cells/μL (20 and 200 cells per cell plate well) were immediately added to a solution containing FET molecular oscillators formed from the oligonucleotide of SEQ ID NO. 1 and topoisomerase SEQ ID NO. 2 at 2 fnol/mL in 100 mM Tris-HCl (pH 7.4). Normal control cells which did not have blunt-ended DNA breaks were used as the control. Donor fluorescence was measured 3 minutes after adding the apoptotic cells having DNA breaks, whereas no such fluorescence was observed in the control. Detection was performed using a Tecan GENios Plus at 485 nm excitation and 535 nm emission wavelengths.

From the foregoing description, one skilled in the art can easily ascertain the essential characteristics of this disclosure, and without departing from the spirit and scope thereof, can make various changes and modifications to adapt the disclosure to various usages and conditions. The embodiments described hereinabove are meant to be illustrative only and should not be taken as limiting of the scope of the disclosures which is defined in the following claims.

What is claimed is:

1. A semi-artificial nanomachine, said nanomachine comprising:
a first biological molecule and
a second biological molecule, wherein said second biological molecule comprises a complementary sequence for binding of said first biological molecule, wherein binding of said first biological molecule to said second biological molecule results in dissociation of the nanomachine into smaller working units, and wherein said smaller working units perform an observable function.

2. The semi-artificial nanomachine of claim 1, wherein the first biological molecule and second biological molecule bind together by self-assembly.

3. A method to detect deoxyribonucleic acid (DNA) breaks, said method comprising the steps of:
a) providing a synthetic oligonucleotide comprising
a topoisomerase recognition sequence;
a fluorescence donor; and,
a fluorescence acceptor;
b) providing a type I topoisomerase capable of binding said topoisomerase recognition sequence;
c) mixing said synthetic oligonucleotide and said topoisomerase and allowing self-assembly of a fluorescence energy transfer (FET) oscillator to occur;
d) adding a DNA sample to the FET oscillator to create a mixture;
e) irradiating said mixture at an absorption wavelength of said fluorescence donor; and,
f) measuring the emission spectrum of said irradiated mixture.

4. The method of claim 3, wherein said fluorescence acceptor comprises a nonradiative fluorescence quencher.

5. The method of claim 3, wherein said fluorescence donor is a fluorescein derivative.

6. The method of claim 3, wherein said fluorescence acceptor is a fluorescein derivative.

7. The method of claim 3, wherein said fluorescence donor is a rhodamine derivative.

8. The method of claim 3, wherein said fluorescence acceptor is a rhodamine derivative.

9. The method of claim 3, wherein said fluorescence acceptor is a fluorescein derivative and said fluorescence acceptor is a rhodamine derivative.

10. The method of claim 3, wherein said synthetic oligonucleotide comprises a nucleotide sequence corresponding to SEQ ID NO. 1.

11. The method of claim 3, wherein said synthetic oligonucleotide comprises a nucleotide sequence corresponding to SEQ ID NO. 6.

12. The method of claim 3, wherein the type 1 topoisomerase is a virus-encoded eukaryotic type IB topoisomerase.

13. The method of claim 12, wherein said virus-encoded eukaryotic type IB topoisomerase comprises a peptide sequence corresponding to SEQ ID NO. 2.

14. A probe for detecting deoxyribonucleic acid (DNA) sequence breaks, said probe comprising:
a synthetic oligonucleotide comprising
a topoisomerase recognition sequence;
a fluorescence donor; and,
a nonradiative fluorescence quencher.

15. The probe of claim 14, further comprising a type I topoisomerase capable of binding to said topoisomerase recognition sequence, wherein the binding results in self-assembly.

16. The probe of claim 15, wherein said type I topoisomerase is a virus-encoded eukaryotic type IB topoisomerase comprising a peptide sequence corresponding to SEQ ID NO. 2.

17. The probe of claim 14, wherein said synthetic oligonucleotide sequence comprises a nucleotide sequence corresponding to SEQ ID NO. 4.

18. A probe for detecting deoxyribonucleic acid (DNA) sequence breaks, said probe comprising:
a synthetic oligonucleotide comprising
a topoisomerase recognition sequence;
a fluorescence donor; and,
a fluorescence acceptor.

19. The probe of claim 18, further comprising a type I topoisomerase capable of binding to said topoisomerase recognition sequence, wherein the binding results in self-assembly.

20. The probe of claim 18, wherein said type 1 topoisomerase is a virus-encoded eukaryotic type 1B topoisomerase comprising a peptide sequence corresponding to SEQ ID NO. 2.

21. The probe of claim 18, wherein said synthetic oligonucleotide sequence comprises a nucleotide sequence corresponding to SEQ ID NO. 5.

22. A method to detect deoxyribonucleic acid (DNA) breaks, said method comprising the steps of:
providing the probe of claim 14;
providing a DNA sample;
irradiating the mixture of said probe and said DNA sample at an absorption wavelength of said fluorescence donor; and,
measuring the emission spectrum of said irradiated mixture.

23. The method of claim 22, wherein the step of providing provides the probe of claim 18.

24. A kit for detecting DNA breaks comprising the FET oscillator of claim 3.

25. A kit for detecting DNA breaks comprising the probe of claim 18.

26. A kit for detecting DNA breaks comprising the probe of claim 14.


SEQUENCE LISTING (6 sequences)

SEQ ID NO: 1 - DNA, 38 bp, Artificial Sequence
  Synthesized by Integrated DNA Technologies (IDT, Coralville, IA)
aagggacctgcngcaggtcccttaacgcatnatgcgtt

SEQ ID NO: 2 - protein, 314 aa, Vaccinia virus
MRALFYKDGKLFTDNNFLNPVSDDNPAYEVLQHVKIPTHLTDVVVYEQTWEEALTRLIFVGSDSKGRRQYFYGKMHVQNRNAKRDRIFVRVYNVMKRINCFINKNIKKSSTDSNYQLAVFMLMETMFFIRFGKMKYLKENETVGLLTLKNKHIEISPDEIVIKFVGKDKVSHEFVVHKSNRLYKPLLKLTDDSSPEEFLFNKLSERKVYECIKQFGIRIKDLRTYGVNYTFLYNFWTNVKSISPLPSPKKLIALTIKQTAEVVGHTPSISKRAYMATTILEMVKDKNFLDVVSKTTFDEFLSIVVDHVKSSTDG

SEQ ID NO: 3 - DNA, 21 bp, Artificial Sequence
  Synthesized by IDT (Integrated DNA Technologies, Coralville, IA)
gcgctagacctggtctagcgc

SEQ ID NO: 4 - DNA, 34 bp, Artificial Sequence
  Synthesized by IDT (Integrated DNA Technologies, Coralville, IA).
nagggacctctgaggtcccttacgcattatgcgn

SEQ ID NO: 5 - DNA, 29 bp, Artificial Sequence
  Synthesized by IDT (Integrated DNA Technologies, Coralville, IA).
aagggactnagtcccttacgattnatcgt

SEQ ID NO: 6 - DNA, 38 bp, Artificial Sequence
  Synthesized by IDT (Integrated DNA Technologies, Coralville, IL)
aagggncctgctgcaggacccttaacgcattatgcgnt